(19) United States
(12) Patent Application Publication
(10) Pub. No.: US 2022/0151733 A1
(43) Pub. Date: May 19, 2022

(54) TOOTH RESTORATION SYSTEM AND METHOD

(71) Applicant: CHENG-KANG CHOU, Taoyuan City (TW)

(72) Inventors: HSIAO-YANG LIU, Taoyuan City (TW); CHENG-KANG CHOU, Taoyuan City (TW); YI-CHEN CHEN, Nantou County (TW); DA-RONG LIU, Tainan City (TW)

(21) Appl. No.: 17/525,989

(22) Filed: Nov. 15, 2021

(30) Foreign Application Priority Data
Nov. 18, 2020 (TW) 109140196

(51) Int. Cl. A61C 5/20; G06T 7/90; A61C 9/00; A61C 19/05; A61C 13/00; A61C 13/20; A61B 1/24

(52) U.S. Cl. CPC A61C 5/20; G06T 7/90; A61C 9/0053; A61C 19/05; A61C 13/0004; A61C 2201/002; A61C 13/20; A61B 1/24; G06T 2207/10024; G06T 2207/30036; A61C 13/0019

(57) ABSTRACT

A tooth restoration method and a tooth restoration system are provided. The tooth restoration method includes: configuring a control module to obtain an image and tooth characteristic information of teeth through an image capturing module; configuring the control module to calculate and generate a characteristic parameter of the target tooth according to the image and the tooth characteristic information, and generate a 3D restoration file of the target tooth according to the characteristic parameter; configuring the control module to calculate and generate color information and candidate filling materials of the target tooth according to the image, the 3D restoration file and the filling material database, and compare the candidate filling materials with the characteristic parameter and the color information of the target tooth to generate a selected filling material matching the target tooth; and restoring the target tooth based on the selected filling material through a prosthetic technique.

### CROSS-REFERENCE TO RELATED APPLICATION

This application claims the priority benefit of Taiwan application (109140196), filed on Nov. 18, 2020. The entirety of the above-mentioned patent application is hereby incorporated by reference herein and made a part of this specification.

### FIELD OF THE INVENTION

The present invention relates to a tooth restoration system and method, and more particularly to a tooth restoration system and method based on a machine learning model.

### BACKGROUND OF THE INVENTION

In the past, when correcting or restoring teeth, patients not only needed to spend a lot of time and money, which caused long-term inconvenience in life, but also had to endure the discomfort during tooth extraction and adapting to the correction process. With the development of dental technology, although the current methods of orthodontics or restoration of teeth can improve some of the previous defects, there are still some defects that need to be improved. For example, there is a clear color difference between the color of the restored tooth and the color of the natural tooth, the shape of the teeth after correction or restoration is significantly different from the shape of the symmetrical teeth or incompatible with the shape of the adjacent teeth, and compared with natural teeth, the aesthetics of the teeth after correction or restoration is still slightly insufficient. Therefore, how to provide a method to avoid the color and shape difference between the teeth after correction or restoration and the natural teeth, as well as the overall aesthetics of the teeth, will be the focus of this invention.

### SUMMARY OF THE INVENTION

In view of this, an embodiment of the present invention provides a tooth restoration system, which includes an image capturing module, a filling material database and a control module. The image capturing module is configured to scan or photograph a plurality of teeth in a patient's mouth, wherein the plurality of teeth include a target tooth. The filling material database is configured to store a plurality pieces of parameter information, wherein the plurality pieces of parameter information include various single-layer filling material color parameter information and parameter information of specific thickness ratio of enamel to dentin. The control module is coupled to the image capturing module and the filling material database and is configured to obtain an image and tooth characteristic information of the plurality of teeth through the image capturing module and obtain the plurality pieces of parameter information through the filling material database. The control module calculates and generates a characteristic parameter of the target tooth according to the image and the tooth characteristic information, and generates a 3D restoration file of the target tooth according to the characteristic parameter. The control module calculates and generates color information and a plurality of candidate filling materials of the target tooth according to the image, the 3D restoration file and the filling material database, and compares the plurality of candidate filling materials with the characteristic parameter and the color information of the target tooth to generate a selected filling material that matches the target tooth. The selected filling material is selected from the plurality of candidate filling materials. The target tooth is restored based on the selected filling material through a prosthetic technique.

In an embodiment of the present invention, a process in which the control module calculates and generates the color information and the plurality of candidate filling materials of the target tooth according to the image, the 3D restoration file and the filling material database includes: configuring the control module to calculate and generate the color information of the target tooth according to the image and the 3D restoration file; and configuring the control module to calculate and generate the plurality of candidate filing materials according to the 3D restoration file, the color information and the filling material database.

In an embodiment of the present invention, the target tooth is a tooth to be repaired or a tooth to be beautified.

In an embodiment of the present invention, the tooth characteristic information includes tooth shape, tooth thickness, tooth color, tooth texture, upper and lower jaw relationship, occlusal distance, tooth length-to-width ratio, symmetry, anterior tooth aesthetic principle and tooth smile curve.

In an embodiment of the present invention, the characteristic parameter includes position, shape and thickness of the target tooth.

In an embodiment of the present invention, the plurality of candidate filling materials are any materials that fill/repair/restore teeth and are fixed on a tooth.

In an embodiment of the present invention, the plurality of candidate filling materials include composite resin, glass ionomer cement, ceramic and zirconia.

In an embodiment of the present invention, the plurality pieces of parameter information in the filling material database include brand, product name, thickness, color under different backgrounds and light sources, transparency and spectrum performance.

In an embodiment of the present invention, the prosthetic technique includes free-hand stacking, layered filling by using guide plates, and directly adhering the selected filling material that is shaped and cured outside a body to the target tooth.

In an embodiment of the present invention, a process in which the target tooth is restored based on the selected filling material through the prosthetic technique includes: filling a first selected filling material into a gap between an inner guide plate and the target tooth; and filling a second selected filling material into a gap between an outer guide plate and the first selected filling material.

In an embodiment of the present invention, a production method of the selected filling material includes 3D printing, manual molding, casting, computer-aided manufacturing (CAM), and computerized numerical control (CNC) cutting.

Another embodiment of the present invention provides a tooth restoration method applicable to a tooth restoration system. The tooth restoration system includes an image capturing module, a filling material database and a control module coupled to the image capturing module and the filling material database. The image capturing module is configured to scan or photograph a plurality of teeth in a patient's mouth, wherein the plurality of teeth include a target tooth. The filling material database is configured to store a plurality pieces of parameter information, wherein the plurality pieces of parameter information include various single-layer filling material color parameter information and parameter information of specific thickness ratio of enamel to dentin. The tooth restoration method includes steps of: configuring the control module to obtain an image and tooth characteristic information of the plurality of teeth through the image capturing module; configuring the control module to calculate and generate a characteristic parameter of the target tooth according to the image and the tooth characteristic information, and generate a 3D restoration file of the target tooth according to the characteristic parameter; configuring the control module to calculate and generate color information and a plurality of candidate filling materials of the target tooth according to the image, the 3D restoration file and the filling material database, and compare the plurality of candidate filling materials with the characteristic parameter and the color information of the target tooth to generate a selected filling material that matches the target tooth, wherein the selected filling material is selected from the plurality of candidate filling materials; and restoring the target tooth based on the selected filling material through a prosthetic technique.

In an embodiment of the present invention, the step of configuring the control module to calculate and generate the color information and the plurality of candidate filling materials of the target tooth according to the image, the 3D restoration file and the filling material database includes steps of: configuring the control module to calculate and generate the color information of the target tooth according to the image and the 3D restoration file; and configuring the control module to calculate and generate the plurality of candidate filing materials according to the 3D restoration file, the color information and the filling material database.

The tooth restoration system and method provided by the embodiments of the present invention perform the following operations: calculating and generating the characteristic parameter of the target tooth according to the image and the tooth characteristic information of the teeth; generating the 3D restoration file of the target tooth according to the characteristic parameter of the target tooth; calculating and generating the color information and the candidate filling materials of the target tooth according to the image of the teeth, the 3D restoration file of the target tooth and the various parameter information stored in the filling material database, and comparing the candidate filling materials with the characteristic parameter and the color information of the target tooth to generate the selected filling material that matches the target tooth; and restoring the target tooth based on the selected filling material through a prosthetic technique. In this way, there is almost no difference in color, shape, and thickness between the restored target tooth and the adjacent natural tooth, thereby greatly improving the overall aesthetics of the tooth.

### BRIEF DESCRIPTION OF THE DRAWINGS

The present invention will become more readily apparent to those ordinarily skilled in the art after reviewing the following detailed description and accompanying drawings, in which:

FIG. 1 is a block diagram of a tooth restoration system according to an embodiment of the present invention;

FIG. 2 is a schematic flowchart of a tooth restoration method according to an embodiment of the present invention;

FIG. 3 is a schematic partial diagram of an image of teeth in a patient's mouth;

FIG. 4 is a schematic diagram of the restored shape of the broken part of the target tooth in FIG. 3 predicted by the tooth restoration method according to an embodiment of the present invention;

FIG. 5 is a schematic flowchart of the process of predicting the color information of the target tooth and generating the selected filling material that matches the target tooth in step S130 of FIG. 2 according to an embodiment of the present invention; and

FIG. 6 is a schematic flowchart of the process of restoring the target tooth based on the selected filling material through the patching technique in step S140 of FIG. 2 according to an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

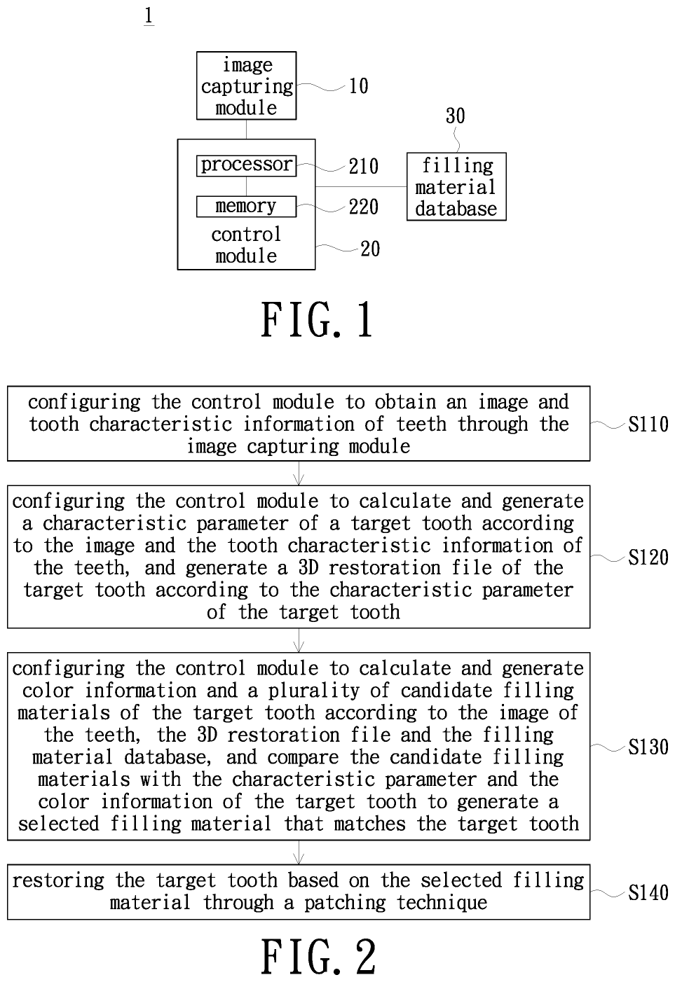

Please refer to FIG. 1, which is a block diagram of a tooth restoration system according to an embodiment of the present invention. The tooth restoration system 1 includes an image capturing module 10, a control module 20 and a filling material database 30. The control module 20 includes a processor 210 and a memory 220. The memory 220 is configured to store a computer program. The processor 210 is configured to read the computer program to execute a tooth restoration method. The image capturing module 10 is configured to scan or photograph all or part of the teeth in the patient's mouth, wherein the teeth may include teeth to be repaired (e.g., caries, broken tooth) or teeth to be beautified (e.g., teeth with too large gaps, nail-shaped teeth). To simplify the description, the tooth to be repaired or beautified is referred to as a target tooth in the following.

The control module 20 is coupled to the image capturing module 10 and the filling material database 30. The control module 20 is configured to obtain or capture the image and the tooth characteristic information of the teeth in the patient's mouth through the image capturing module 10. The tooth characteristic information includes tooth shape, tooth thickness, tooth color, tooth texture, upper and lower jaw relationship, occlusal distance, tooth length-to-width ratio, symmetry, anterior tooth aesthetic principle and tooth smile curve. Preferably, the tooth smile curve is defined that the line of the cut ends of the upper teeth is similar to the curvature of the upper edge of the lower lip when smiling. In addition, the control module 20 is further configured to obtain, through the filling material database 30, various parameter information, including the brand, product name, thickness, color under different backgrounds and light sources, transparency and spectrum performance of filling materials. In addition, the parameter information can also include various single-layer resin color parameter information and the parameter information of specific thickness ratio of enamel to dentin that is common in clinical practice. The various single-layer resin color parameter information includes the single-layer resin color parameter information of different brands, different series, different colors and different thicknesses.

In an embodiment of the present invention, the image capturing module 10 may be a digital oral scanner or a camera. The processor 210 may be a central processing unit (CPU), a graphics processing unit (GPU), an application-specific integrated circuit (ASIC), a distributed processing system, or a suitable processing circuit. The memory 220 is preferably a non-transitory computer-readable storage medium, such as a hard disk, solid-state memory, random access memory (RAM), and read-only memory (ROM). In some embodiments, the memory 120 may be further configured to store related algorithms corresponding to machine learning models or virtual data and related algorithms corresponding to deep learning models. The deep learning model uses artificial intelligence (AI) technology to implement neural networks (e.g., convolutional neural networks) and is pre-trained by a large amount of data, so that the deep learning model after training can perform an accurate prediction, such as predicting the color, shape, and thickness of the target tooth, and the type, shape, thickness, and color number of the filling material that matches the target tooth.

Next, please refer to FIG. 2, which is a schematic flowchart of a tooth restoration method according to an embodiment of the present invention. The tooth restoration method is applicable to the tooth restoration system 1 and includes steps S110 to S140. First, step S110: configuring the control module 20 to obtain the image and the tooth characteristic information of the teeth through the image capturing module 10. Next, step S120: configuring the control module 20 to calculate and generate the characteristic parameter of the target tooth according to the image and the tooth characteristic information of the teeth, and generate a 3D restoration file of the target tooth according to the characteristic parameter of the target tooth. The characteristic parameter of the target tooth includes the position, shape and thickness of the target tooth, but is not limited thereto.

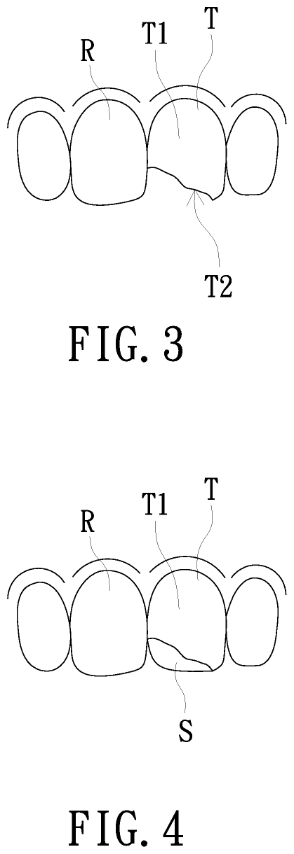

FIGS. 3 and 4 are used to further explain the operation process in step S120 of FIG. 2. FIG. 3 is a schematic partial diagram of an image of teeth in a patient's mouth, and FIG. 4 is a schematic diagram of the restored shape of the broken part of the target tooth in FIG. 3 predicted by the tooth restoration method according to an embodiment of the present invention. Herein, it is supposed that the target tooth T is the right one of the upper front incisor, and the symptom is that a part of the target tooth T is broken. In an embodiment of the present invention, the control module 20 predicts the 3D restoration shape of the broken part T2 of the target tooth T and generates a prediction result (the symbol S in FIG. 4 is the prediction result of the 2D restoration shape) according to the image and the tooth characteristic information of the teeth (that is, the image and tooth characteristic information of the remaining part T1 of the target tooth T (i.e., the original and unbroken part)) and the image and the tooth characteristic information of the symmetrical tooth R of the tooth T (that is, the left one of the upper front incisor) based on the shape prediction algorithm of the deep learning model after training, and performs the 3D digital restoration of the broken part T2 of the target tooth T according to the prediction result.

In another embodiment of the present invention, in addition to predicting the 3D restoration shape of the broken part T2 of the target tooth T according to the image and the tooth characteristic information of the teeth based on the shape prediction algorithm of the deep learning model after training, the control module 20 further automatically designs the 3D digital restoration of the contour of the target tooth T that meets the specific aesthetic conditions according to the anterior tooth aesthetic principle and/or the tooth smile curve.

Next, step S130: configuring the control module 20 to calculate and generate color information (e.g., partial color, overall color, gradual change of color) and a plurality of candidate filling materials of the target tooth T according to the image of the teeth, the 3D restoration file of the target tooth T and the filling material database 30, and compare the candidate filling materials with the characteristic parameter and the color information of the target tooth T to generate a selected filling material that matches the target tooth T. The characteristic parameter of the target tooth T includes the position, shape and thickness of the target tooth T, and the selected filling material is selected from the candidate filling materials. In an embodiment of the present invention, the candidate filling materials and the selected filling material can be any material that can be used to fill, repair, or restore teeth and is fixed on the tooth, such as composite resin, glass ionomer cement, ceramic and zirconia. In addition, the production methods of the selected filling material are, for example, 3D printing, manual molding, casting, computer-aided manufacturing (CAM), and computerized numerical control (CNC) cutting.

More specifically, the control module 20 predicts the color of the broken part T2 of the target tooth T and generates a corresponding prediction result as well as predicting the candidate filling materials matching the broken part T2 of the target tooth T and generating a corresponding prediction result according to the color information of the remaining part T1 of the target tooth T, the 3D restoration shape of the broken part T2 of the target tooth T and the filling material database 30 storing various parameter information based on the color prediction algorithm and the candidate filling material prediction algorithm of the deep learning model after training.

Regarding the color performance of the candidate filling materials, in an embodiment of the present invention, several color detection technologies and optical models (e.g., Lab color space or its equivalent color space, three primary color light mode, Alpha synthesis, etc.) can be used to obtain the single-layer resin color parameters of different brands, different series, different colors, and different thicknesses, and the specific thickness ratio of enamel to dentin that is common in clinical practice is collected, thereby obtaining the color performances of the candidate filling materials before and after superimposing by forward and reverse engineering.

Then, based on the selected filling material prediction algorithm of the deep learning model after training, the candidate filling materials recommended by the prediction result are compared with the characteristic parameter and the color information of the target tooth T (such as style comparison) to generate the selected filling material that matches the target tooth T. The candidate filling materials and the selected filling material can be a filling material of a specific color (including overall color, gradual change of color), shape, and thickness formed by superimposing a plurality of single-layer filling materials of different types and styles. In addition, there can be only one or more selected filling materials. In an embodiment of the present invention, the filling material is preferably a resin, including various enamel resins and dentin resins, but not limited thereto. The filling material database 30 is preferably a resin database with a large number of resin superimposed results.

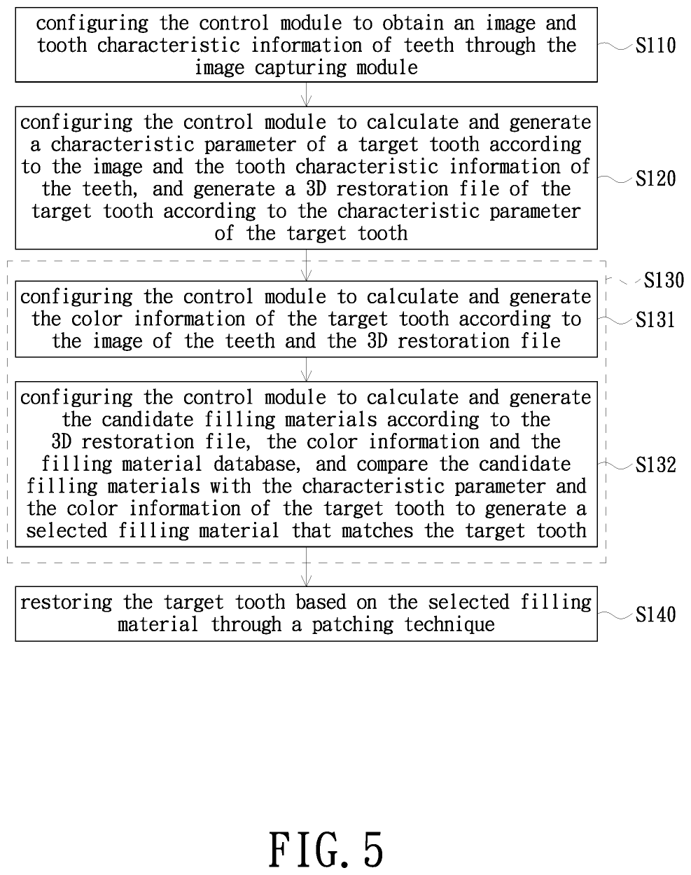

FIG. 5 is used to further explain the operation process of step S130 in FIG. 2. Please refer to FIG. 5, which is a schematic flowchart of the process of predicting the color information of the target tooth and generating the selected filling material that matches the target tooth in step S130 of FIG. 2 according to an embodiment of the present invention. In an embodiment of the present invention, step S130 further includes steps S131 and S132. Step S131: configuring the control module 20 to calculate and generate the color information of the target tooth T according to the image of the teeth and the 3D restoration file of the target tooth T. Furthermore, the control module 20 predicts the color of the broken part T2 of the target tooth T according to the color information of the remaining part T1 of the target tooth T and the 3D restoration shape of the broken part T2 of the target tooth T based on the color prediction algorithm of the deep learning model after training.

In some embodiments, the control module 20 predicts the color of the broken part T2 of the target tooth T according to the color information of the remaining part T1 of the target tooth T, the color information of the teeth adjacent to the target tooth T and/or the color information of the symmetrical teeth R of the target tooth T, and the 3D restoration shape of the broken part T2 of the target tooth T based on the color prediction algorithm of the deep learning model after training to generate the prediction result, that is, generate the color information of the broken part T2 of the target tooth T.

Next. step S132: configuring the control module 20 to calculate and generate the candidate filling materials according to the 3D restoration file, the color information and the filling material database 30, and compare the candidate filling materials with the characteristic parameter and the color information of the target tooth T to generate the selected filling material that matches the target tooth T. The selected filling material is selected from the candidate filling materials.

More specifically, the control module 20 generates the candidate filling materials that matches the broken part T2 of the target tooth T according to the 3D predicted restoration shape of the broken part T2 of the target tooth T, the color information of the remaining part T1 of the target tooth T (i.e., the result predicted in step S131) and the various parameter information stored in the filling material database 30 based on the candidate filling material prediction algorithm of the deep learning model after training. Then, the control module 20 compares the candidate filling materials with the characteristic parameter and the color information of the target tooth T based on the candidate filling material prediction algorithm of the deep learning model after training to generate the selected filling material that matches the target tooth T (i.e., providing recommendations for the best filling material). The selected filling material includes the first-layer filling material (i.e., the inner-layer or bottom enamel filling material), the second-layer filling material (i.e., the dentin filling material) and the third-layer filling material (i.e., the outer enamel filling material), or includes the inner-layer selected filling material (i.e., the dentin filling material) and the outer selected filling material (i.e., the enamel filling material). In other words, there is almost no color difference between the color of the selected filling material and the color of the remaining part T1 of the target tooth T and/or the color of the symmetrical tooth R. The shape and thickness of the selected filling material is almost the same as those of the part of the symmetrical tooth R corresponding to the broken part T2 (i.e., corresponding to the broken part T2 of the target tooth T).

Next, step S140: restoring the target tooth T based on the selected filling material through the patching technique. In an embodiment of the present invention, the prosthetic technique includes free-hand stacking, layered filling by using guide plates, and directly adhering the selected filling material that is shaped and cured outside the body to the target tooth, but it is not limited thereto. For example, the dentist can use a guide plate to fill the patient's target tooth T in layers based on the selected filling material to restore the target tooth T. In an embodiment, the first-layer filling material can use a first guide plate (e.g., tongue side guide plate), the second-layer filling material can use a second guide plate (e.g., dentin guide plate), and the third-layer filling material can use a third guide plate (e.g., labial guide plate). In another embodiment, the dentin filling material can use an inner guide plate, and the enamel filling material can use an outer guide plate. In addition, it should be noted that the aforementioned guide plate is made by using a vacuum suction thermoplastic sheet to form a 3D dental mold sleeve that can be filled with a filling material. As described above, there is almost no color difference between the color of the selected filling material and the color of the remaining part T1 of the target tooth T and/or the color of the symmetrical tooth R, and the shape and thickness of the selected filling material is almost the same as those of the part of the symmetrical tooth R corresponding to the broken part T2 (i.e., corresponding to the broken part T2 of the target tooth T). As a result, there is almost no difference in color, shape, and thickness between the restored target tooth T and the adjacent natural tooth, thereby greatly improving the overall aesthetics of the tooth. In another embodiment of the present invention, the dentist can print a 3D dental model through 3D printing based on the selected filling material and cut the 3D dental model through the computer, so that the dentist can perform the filling according to the 3D dental model.

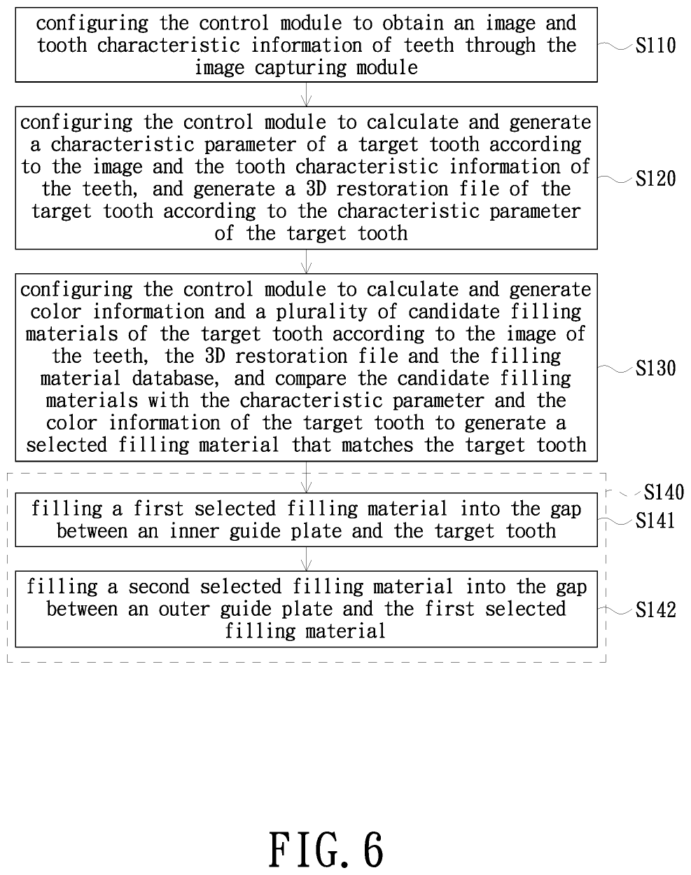

FIG. 6 is used to further explain the operation process of step S140 in FIG. 2. Please refer to FIG. 6, which is a schematic flowchart of the process of restoring the target tooth based on the selected filling material through the patching technique in step S140 of FIG. 2 according to an embodiment of the present invention. In an embodiment of the present invention, step S140 further includes steps S141 and S142 when the control module 20 uses the inner guide plate and the outer guide plate to fill the target tooth T of the patient in layers based on the selected filling material. Step S141: filling the inner-layer selected filling material (i.e., the dentin filling material) into the gap between the inner guide plate and the target tooth T. Next, step S142: filling the outer selected filling material (i.e., the enamel filling material) into the gap between the outer guide plate and the inner selected filling material. Thus, there is almost no difference in color, shape, and thickness between the restored target tooth T and the adjacent natural tooth, thereby greatly improving the overall aesthetics of the tooth.

In summary, the tooth restoration system and method provided by the embodiments of the present invention perform the following operations: calculating and generating the characteristic parameter of the target tooth according to the image and the tooth characteristic information of the teeth; generating the 3D restoration file of the target tooth according to the characteristic parameter of the target tooth; calculating and generating the color information and the candidate filling materials of the target tooth according to the image of the teeth, the 3D restoration file of the target tooth and the various parameter information stored in the filling material database, and comparing the candidate filling materials with the characteristic parameter and the color information of the target tooth to generate the selected filling material that matches the target tooth; and restoring the target tooth based on the selected filling material through a prosthetic technique. In this way, there is almost no difference in color, shape, and thickness between the restored target tooth T and the adjacent natural tooth, thereby greatly improving the overall aesthetics of the tooth.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A tooth restoration system, comprising:
an image capturing module, configured to scan or photograph a plurality of teeth in a patient's mouth, wherein the plurality of teeth comprise a target tooth;
a filling material database, configured to store a plurality pieces of parameter information, wherein the plurality pieces of parameter information comprise various single-layer filling material color parameter information and parameter information of specific thickness ratio of enamel to dentin; and
a control module, coupled to the image capturing module and the filling material database, configured to obtain an image and tooth characteristic information of the plurality of teeth through the image capturing module and obtain the plurality pieces of parameter information through the filling material database;
wherein the control module calculates and generates a characteristic parameter of the target tooth according to the image and the tooth characteristic information, and generates a 3D restoration file of the target tooth according to the characteristic parameter,
wherein the control module calculates and generates color information and a plurality of candidate filling materials of the target tooth according to the image, the 3D restoration file and the filling material database, and compares the plurality of candidate filling materials with the characteristic parameter and the color information of the target tooth to generate a selected filling material that matches the target tooth, wherein the selected filling material is selected from the plurality of candidate filling materials,
wherein, the target tooth is restored based on the selected filling material through a prosthetic technique.

2. The tooth restoration system according to claim 1, wherein a process in which the control module calculates and generates the color information and the plurality of candidate filling materials of the target tooth according to the image, the 3D restoration file and the filling material database comprises:
configuring the control module to calculate and generate the color information of the target tooth according to the image and the 3D restoration file; and
configuring the control module to calculate and generate the plurality of candidate filing materials according to the 3D restoration file, the color information and the filling material database.

3. The tooth restoration system according to claim 1, wherein the target tooth is a tooth to be repaired or a tooth to be beautified.

4. The tooth restoration system according to claim 1, wherein the tooth characteristic information comprises tooth shape, tooth thickness, tooth color, tooth texture, upper and lower jaw relationship, occlusal distance, tooth length-to-width ratio, symmetry, anterior tooth aesthetic principle and tooth smile curve.

5. The tooth restoration system according to claim 1, wherein the characteristic parameter comprises position, shape and thickness of the target tooth.

6. The tooth restoration system according to claim 1, wherein the plurality of candidate filling materials are any materials that fill/repair/restore teeth and are fixed on a tooth.

7. The tooth restoration system according to claim 1, wherein the plurality of candidate filling materials comprise composite resin, glass ionomer cement, ceramic and zirconia, and wherein a production method of the selected filling material comprises 3D printing, manual molding, casting, computer-aided manufacturing (CAM), and computerized numerical control (CNC) cutting.

8. The tooth restoration system according to claim 1, wherein the plurality pieces of parameter information in the filling material database comprise brand, product name, thickness, color under different backgrounds and light sources, transparency and spectrum performance.

9. The tooth restoration system according to claim 1, wherein the prosthetic technique comprises free-hand stacking, layered filling by using guide plates, and directly adhering the selected filling material that is shaped and cured outside a body to the target tooth.

10. The tooth restoration system according to claim 9, wherein a process in which the target tooth is restored based on the selected filling material through the prosthetic technique comprises:
filling a first selected filling material into a gap between an inner guide plate and the target tooth; and
filling a second selected filling material into a gap between an outer guide plate and the first selected filling material.

11. A tooth restoration method applicable to a tooth restoration system, the tooth restoration system comprising an image capturing module, a filling material database and a control module coupled to the image capturing module and the filling material database, the control module being configured to scan or photograph a plurality of teeth in a patient's mouth, the plurality of teeth comprising a target tooth, the filling material database being configured to store a plurality pieces of parameter information, the plurality pieces of parameter information comprising various single-layer filling material color parameter information and parameter information of specific thickness ratio of enamel to dentin, and the tooth restoration method comprising steps of:
configuring the control module to obtain an image and tooth characteristic information of the plurality of teeth through the image capturing module;
configuring the control module to calculate and generate a characteristic parameter of the target tooth according to the image and the tooth characteristic information, and generate a 3D restoration file of the target tooth according to the characteristic parameter;
configuring the control module to calculate and generate color information and a plurality of candidate filling materials of the target tooth according to the image, the 3D restoration file and the filling material database, and compare the plurality of candidate filling materials with the characteristic parameter and the color information of the target tooth to generate a selected filling material that matches the target tooth, wherein the selected filling material is selected from the plurality of candidate filling materials; and
restoring the target tooth based on the selected filling material through a prosthetic technique.

12. The tooth restoration method according to claim 11, wherein the step of configuring the control module to calculate and generate the color information and the plurality of candidate filling materials of the target tooth according to the image, the 3D restoration file and the filling material database comprises steps of:
configuring the control module to calculate and generate the color information of the target tooth according to the image and the 3D restoration file; and
configuring the control module to calculate and generate the plurality of candidate filing materials according to the 3D restoration file, the color information and the filling material database.

13. The tooth restoration method according to claim 11, wherein the target tooth is a tooth to be repaired or a tooth to be beautified.

14. The tooth restoration method according to claim 11, wherein the tooth characteristic information comprises tooth shape, tooth thickness, tooth color, tooth texture, upper and lower jaw relationship, occlusal distance, tooth length-to-width ratio, symmetry, anterior tooth aesthetic principle and tooth smile curve.

15. The tooth restoration method according to claim 11, wherein the characteristic parameter comprises position, shape and thickness of the target tooth.

16. The tooth restoration method according to claim 11, wherein the plurality of candidate filling materials are any materials that fill/repair/restore teeth and are fixed on a tooth.

17. The tooth restoration method according to claim 11, wherein the plurality of candidate filling materials comprise composite resin, glass ionomer cement, ceramic and zirconia, and wherein a production method of the selected filling material comprises 3D printing, manual molding, casting, computer-aided manufacturing (CAM), and computerized numerical control (CNC) cutting.

18. The tooth restoration method according to claim 11, wherein the plurality pieces of parameter information in the filling material database comprise brand, product name, thickness, color under different backgrounds and light sources, transparency and spectrum performance.

19. The tooth restoration method according to claim 11, wherein the prosthetic technique comprises free-hand stacking, layered filling by using guide plates, and directly adhering the selected filling material that is shaped and cured outside a body to the target tooth.

20. The tooth restoration method according to claim 19, wherein the step of restoring the target tooth based on the selected filling material through the prosthetic technique comprises steps of:
filling a first selected filling material into a gap between an inner guide plate and the target tooth; and
filling a second selected filling material into a gap between an outer guide plate and the first selected filling material.
